(19) United States
(12) Patent Application Publication
(10) Pub. No.: US 2016/0215312 A1
(43) Pub. Date: Jul. 28, 2016

(54) SYSTEMS AND METHODS FOR CONVERTING LIGNOCELLULOSIC BIOMASS TO CONCENTRATED SUGARS

(71) Applicants: Mark T. Holtzapple, College Station, TX (US); Agustin N. Zentay, Spring, TX (US); Melinda E. Wales, Bryan, TX (US)

(72) Inventors: Mark T. Holtzapple, College Station, TX (US); Agustin N. Zentay, Spring, TX (US); Melinda E. Wales, Bryan, TX (US)

(73) Assignee: The Texas A&M University System, College Station, TX (US)

(21) Appl. No.: 15/089,444

(22) Filed: Apr. 1, 2016

Related U.S. Application Data
(63) Continuation of application No. PCT/US2014/058356, filed on Sep. 30, 2014.
(60) Provisional application No. 61/885,345, filed on Oct. 1, 2013.

(51) Int. Cl. C12P 19/02

(52) U.S. Cl. CPC C12P 19/02

(57) ABSTRACT

Various systems and methods are provided for converting lignocellulosic biomass to concentrated sugars. For example, a saccharification process includes obtaining lignocellulosic biomass and converting the lignocellulosic biomass into sugars. The lignocellulosic biomass is converted into sugars by flowing the lignocellulosic biomass countercurrently to a flow of water through a saccharification vessel having a column and converting the lignocellulosic biomass into a sugar solution using an enzyme in the column.

### CROSS-REFERENCE TO RELATED APPLICATIONS AND PRIORITY CLAIM

This application is a continuation of International Patent Application No. PCT/US2014/058356 filed on Sep. 30, 2014, which claims priority to U.S. Provisional Patent Application No. 61/885,345 filed on Oct. 1, 2013. Both of these applications are hereby incorporated by reference in their entirety.

### TECHNICAL FIELD

This disclosure is directed generally to systems and methods for converting biomass to sugars. More specifically, this disclosure is directed to systems and methods for converting lignocellulosic biomass to concentrated sugars.

### BACKGROUND

Lignocellulose refers to dry plant matter or biomass and is often referred to as “lignocellulosic biomass.” Lignocellulosic biomass is one of the most abundant materials available for generating bio-fuels, such as bio-ethanol. There are various forms of lignocellulosic biomass, including virgin lignocellulosic biomass, waste lignocellulosic biomass, and energy crop lignocellulosic biomass. Virgin lignocellulosic biomass includes naturally occurring biomass, such as dried trees, bushes, grass, and other plants. Waste lignocellulosic biomass includes biomass generated as a by-product in various industries, such as biomass from farms or paper mills. Energy crop lignocellulosic biomass includes biomass grown specifically to serve as raw materials for the production of bio-fuels.

### SUMMARY

This disclosure provides systems and methods for converting lignocellulosic biomass to concentrated sugars.

In a first embodiment, a saccharification process includes obtaining lignocellulosic biomass and converting the lignocellulosic biomass into sugars. The lignocellulosic biomass is converted into sugars by flowing the lignocellulosic biomass countercurrently to a flow of water through a saccharification vessel having a column and converting the lignocellulosic biomass into a sugar solution using an enzyme in the column.

In a second embodiment, a saccharification process includes obtaining lignocellulosic biomass and converting the lignocellulosic biomass into sugars. The lignocellulosic biomass is converted into sugars by flowing the lignocellulosic biomass countercurrently to a flow of water through multiple saccharification vessels including a series of stirred tanks, separating solids exiting each stirred tank from sugars exiting each stirred tank, and converting the lignocellulosic biomass into a sugar solution using an enzyme in at least some of the stirred tanks.

In a third embodiment, a saccharification process includes obtaining lignocellulosic biomass and converting the lignocellulosic biomass into sugars. The lignocellulosic biomass is converted into sugars by adding one or more antimicrobial agents to the lignocellulosic biomass and flowing the lignocellulosic biomass countercurrently to a flow of water through at least one saccharification vessel. The lignocellulosic biomass is converted into sugars also by converting the lignocellulosic biomass into a sugar solution using an enzyme in the at least one saccharification vessel. The lignocellulosic biomass is converted into sugars further by concentrating the sugar solution and removing at least a portion of the one or more antimicrobial agents from the sugar solution while concentrating the sugar solution.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 illustrates an example saccharification process according to this disclosure;

FIG. 2 illustrates an example option for achieving countercurrent saccharification according to this disclosure;

FIG. 3 illustrates an example option for achieving countercurrent flow in a packed column according to this disclosure;

FIGS. 4 and 5 illustrate example vapor-compression concentrator systems according to this disclosure;

FIG. 6 illustrates an example multi-effect evaporator system according to this disclosure;

FIGS. 7A through 7D illustrate an example individual heat exchanger tube according to this disclosure;

FIGS. 8A through 8F illustrate example methods for joining a heat exchanger tube to a tube sheet according to this disclosure;

FIGS. 9A through 9E illustrate an example assembled heat exchanger that employs multiple heat exchanger tubes according to this disclosure;

FIGS. 10A through 12B illustrate example jet ejector designs according to this disclosure;

FIG. 13 illustrates an example glucose concentration as a function of time according to this disclosure;

FIGS. 14 and 15 illustrate example glucose and xylose concentrations in each bottle of a first train of a saccharification process as a function of time according to this disclosure;

FIGS. 16 and 17 illustrate example glucose and xylose concentrations in each bottle of a second train of a saccharification process as a function of time according to this disclosure;

FIG. 18 illustrates example total solids in, total glucose out, and total xylose out for an entire experiment in the first train of a saccharification process according to this disclosure;

FIG. 19 illustrates example total solids in, total glucose out, and total xylose out for a steady-state portion of the experiment in the first train of a saccharification process according to this disclosure;

FIG. 20 illustrates example total solids in, total glucose out, and total xylose out for an entire experiment in the second train of a saccharification process according to this disclosure;

FIG. 21 illustrates example total solids in, total glucose out, and total xylose out for a steady-state portion of the experiment in the second train of a saccharification process according to this disclosure; and

FIG. 22 illustrates example conversions achieved in continuous countercurrent saccharification to batch according to this disclosure.

### DETAILED DESCRIPTION

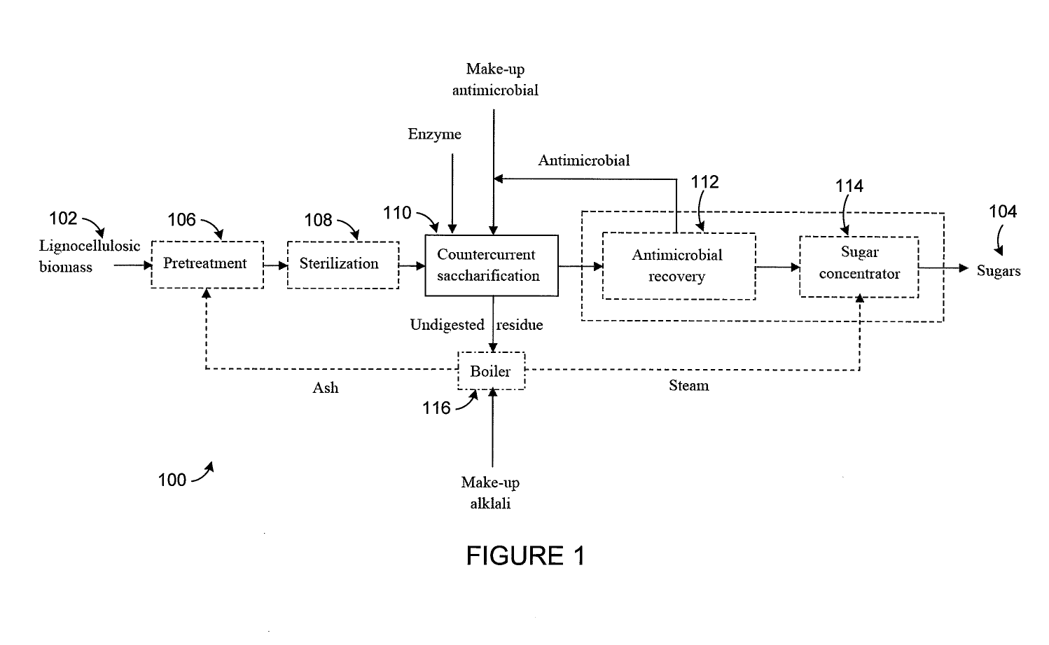

FIGS. 1 through 22, discussed below, and the various embodiments used to describe the principles of this disclosure in this patent document are by way of illustration only and should not be construed in any way to limit the scope of the disclosure. Those skilled in the art will understand that the principles of this disclosure may be implemented in any suitably arranged system. Additionally, the drawings are not necessarily to scale.

FIG. 1 illustrates an example saccharification process 100 according to this disclosure. As shown in FIG. 1, the saccharification process 100 operates to convert lignocellulosic biomass 102 into concentrated sugars 104. The lignocellulosic biomass 102 generally denotes any suitable type(s) of lignocellulosic biomass. The concentrated sugars 104 generally denote any suitable type(s) of sugar(s), such as glucose and xylose.

The saccharification process 100 here includes a pretreatment stage 106, a sterilization stage 108, a countercurrent saccharification stage 110, an antimicrobial recovery stage 112, a sugar concentrator stage 114, and a boiler stage 116. Note that dotted stages in FIG. 1 represent optional stages within the saccharification process 100 that may or may not be used. Also note that the saccharification stage 110 is described as “countercurrent” because liquids flow in one direction while solids flow in the opposite direction.

The pretreatment stage 106 generally operates to pretreat the lignocellulosic biomass 102 in order to allow or enhance enzymatic digestibility of the biomass 102. The pretreatment stage 106 is optional because the lignocellulosic biomass 102 could already be pretreated, and additional pretreatment may not be needed. This may occur, for example, when the lignocellulosic biomass 102 represents paper fines from paper mills. On the other hand, if the lignocellulosic biomass 102 is raw, the lignocellulosic biomass 102 may need pretreatment.

The pretreatment stage 106 could support any suitable operations to pretreat lignocellulosic biomass 102 in order to allow or enhance enzymatic digestibility of the biomass 102. As non-limiting examples, chemical pretreatment technologies include the use of dilute acid, ammonia fiber expansion (AFEX), soaking in aqueous ammonia (SAA), liquid hot water, steam explosion, organo-solvents, ionic liquids, and alkaline treatments.

As a specific example, some embodiments of the pretreatment stage 106 employ oxidative alkalis, such as calcium hydroxide, magnesium hydroxide, sodium hydroxide, or potassium hydroxide. The source of the alkali can be industrial suppliers, such as lime kilns. Alternatively, the alkali can be obtained from boiler ash. The alkali can be supplemented with oxidative reagents, such as air, oxygen, ozone, or hydrogen peroxide. Below are some examples of how lime can be used as the alkali in the pretreatment stage 106:

Short-term nonoxidative lime: Lignocellulose is contacted with lime and water at a temperature of between 70° C. to 120° C. for a time between 1 to 10 hours.

Short-term oxidative lime: Lignocellulose is contacted with lime, water, and oxygen at a temperature of between 70° C. to 180° C. for a time between 1 to 10 hours at a pressure of between 1 to 25 bar.

Long-term nonoxidative lime: Lignocellulose is contacted with lime and water at a temperature of between 25° C. to 100° C. for a time between 15 to 40 days.

Long-term oxidative lime: Lignocellulose is contacted with lime, water, and air at a temperature of between 25° C. to 90° C. for a time between 15 to 40 days at a pressure of 1 bar.

In all of these cases, the lime loading can be 0.05 to 0.3 g Ca(OH)₂/g biomass. Lower lime loadings can be sufficient for non-aggressive (such as short low-temperature) conditions, whereas higher lime loadings may be needed for aggressive (such as long high-temperature) conditions. Sufficient water can be added to ensure good contact of lime and biomass, and water loadings above 10 g H₂O/g biomass are typically sufficient.

In some embodiments, chemical pretreatment methods can be combined with shock pretreatment in the pretreatment stage 106. Shock pretreatment has been shown to be particularly effective at dilute enzyme loadings, which is synergistically compatible with the countercurrent saccharification system.

The sterilization stage 108 generally operates to render the lignocellulosic biomass 102 sterile. The sterilization stage 108 is optional because some pretreatments (such as those using dilute acid or steam explosion) are very severe and render the biomass 102 sterile. If a less severe pretreatment is employed, an additional sterilization step can be provided by the sterilization stage 108. The sterilization stage 108 could support any suitable operations to sterilize biomass 102. In some embodiments, the sterilization stage 108 can sterilize the biomass 102 with high-temperature steam.

The countercurrent saccharification stage 110 generally operates to convert biomass 102 into sugars, such as by converting polysaccharides to sugar dimers and monomers. The sugars are typically contained in a liquid, which is referred to below as a sugar solution. The lignocellulosic biomass 102 here flows countercurrently to a flow of water, which may contain a buffer to maintain pH. Additionally, the flow of water may contain one or more antimicrobial agents that suppress the growth of potential contaminants. At least one hydrolytic enzyme is added to a saccharification vessel within the countercurrent saccharification stage 110, which converts the biomass 102 into sugars. Additional details regarding the countercurrent saccharification stage 110 are provided below.

As shown in FIG. 1, one or more antimicrobial agents can also be added to the countercurrent saccharification stage 110. The antimicrobial agents could come from the antimicrobial recovery stage 112 or as fresh (make-up) agent. Examples of antimicrobial agents include non-volatile agents such as penicillin, tetracycline, sodium azide, and thimerosal. Examples of antimicrobial agents also include volatile agents such as alcohols (like methanol or ethanol), chloroform, phenol, formaldehyde, glutaraldehyde, nonanalm, benzothiazole, or 2-ethyl-1-hexanol.

Various “essential oils” from plants can also have antimicrobial activity. An example is Carum capticum seeds (37% thymol, 36% gamma-terpinen, and 21% cymene). The essential oils from plants such as oregano (Origanum vulgare), clove (Syzygium aromaticum), and thyme (Thymus vulgaris) could be effective. Specific essential oils shown to be effective include carvacrol, citral, eugenol, geranyl acetate, linalool, and thujone. In particular embodiments, it is not necessary to extract essential oils from plants. Instead, plants that contain essential oils can be added directly to a saccharification vessel in the countercurrent saccharification stage 110, which eliminates the cost of extraction and simultaneously serves as a lignocellulose source.

The antimicrobial recovery stage 112 generally operates to remove antimicrobial agents from the generated sugar solution. The antimicrobial recovery stage 112 is optional because there may be no need or desire to remove the antimicrobial agents from the sugar solution. The antimicrobial recovery stage 112 can support any suitable technique for removing antimicrobial agents from sugars. In some embodiments, if the antimicrobial agents are nonvolatile large molecules, they can be readily recovered using membranes with the appropriate size exclusion to pass sugars but retain the antimicrobial agents. If the antimicrobial agents are volatile, they can be readily recovered via evaporation (in which case the recovery of volatile antimicrobials and a sugar concentration operation described below can occur in the same step).

The sugar concentrator stage 114 generally operates to concentrate the sugar solution from the saccharification vessel. The sugar concentrator stage 114 is optional because there may be no need to concentrate the sugar solution from the countercurrent saccharification stage 110. The sugar concentrator stage 114 can support any suitable technique for concentrating sugars. For example, the sugar solution can be concentrated via non-thermal methods, such as reverse osmosis. Alternatively, it is possible to employ thermal methods, such as vapor compression, multi-effect evaporation, and multi-stage flash, to concentrate the sugar solution. Because sugar solutions can be damaged at high temperatures, thermal concentration methods could be performed at lower temperatures, such as less than approximately 100° C. With this temperature limitation, vapor density is low, so mechanical compressors might be very large and costly. One example of an economical alternative involves the use of jet ejectors, which can process large volumes of vapor cost-effectively. Example jet ejector designs are provided below.

The boiler stage 116 can be used to support various functions in the saccharification process 100. For example, dewatered and washed undigested biomass 102 can be sent to the boiler stage 116 to be burned for process heat. The ash that is naturally present in biomass is alkaline and can be used by the pretreatment stage 106 as described above, assuming the pretreatment stage 106 implements an alkaline treatment. Note that make-up alkali-containing salts (such as calcium carbonate) can be added to the boiler stage 116 to supplement the alkalinity of the ash. As another example, the boiler stage 116 can supply steam to the sugar concentrator stage 114 for use in concentrating the sugar solution.

Each stage 106-116 in FIG. 1 could be implemented using any suitable structure(s) for performing the described functions. Example implementations of the countercurrent saccharification stage 110 and sugar concentrator stage 114 are provided below. However, these examples are for illustration only and could be implemented in any other suitable manner. Moreover, the other stages 106-108, 112, 116 could be implemented in any desired manner.

As noted above, the lignocellulosic biomass 102 is saccharified using hydrolytic enzymes, such as cellulase, hemicellulase, or beta-glucosidase, in the countercurrent saccharification stage 110. Traditional methods for saccharifying biomass employ batch methods where enzymes and biomass are added at the beginning of a reaction. As sugars are released, the sugars accumulate in liquid, which inhibits the enzymes. Also, as the saccharification proceeds, the biomass becomes less reactive because easy-to-digest portions of the biomass saccharify first. The consequence is that, in the later portions of the saccharification process, the biomass is less reactive and product inhibition is strong. Because of that, reaction rates approach zero near the end of the reaction, which limits the ultimate conversion.

Countercurrent saccharification in the countercurrent saccharification stage 110 achieves high biomass conversions with small amounts of enzyme. This is accomplished for various reasons. For example, in regions where the biomass 102 is highly digested (and hence less reactive), sugar concentrations are low and therefore there is less inhibition. In regions where sugar concentrations are high (and hence highly inhibitory), the biomass 102 is less digested and therefore highly reactive. The consequence is that no region of the saccharification has both low-reactivity biomass and high-concentration inhibitory sugars, so high conversions are possible. Further, the sugars are produced at high concentrations.

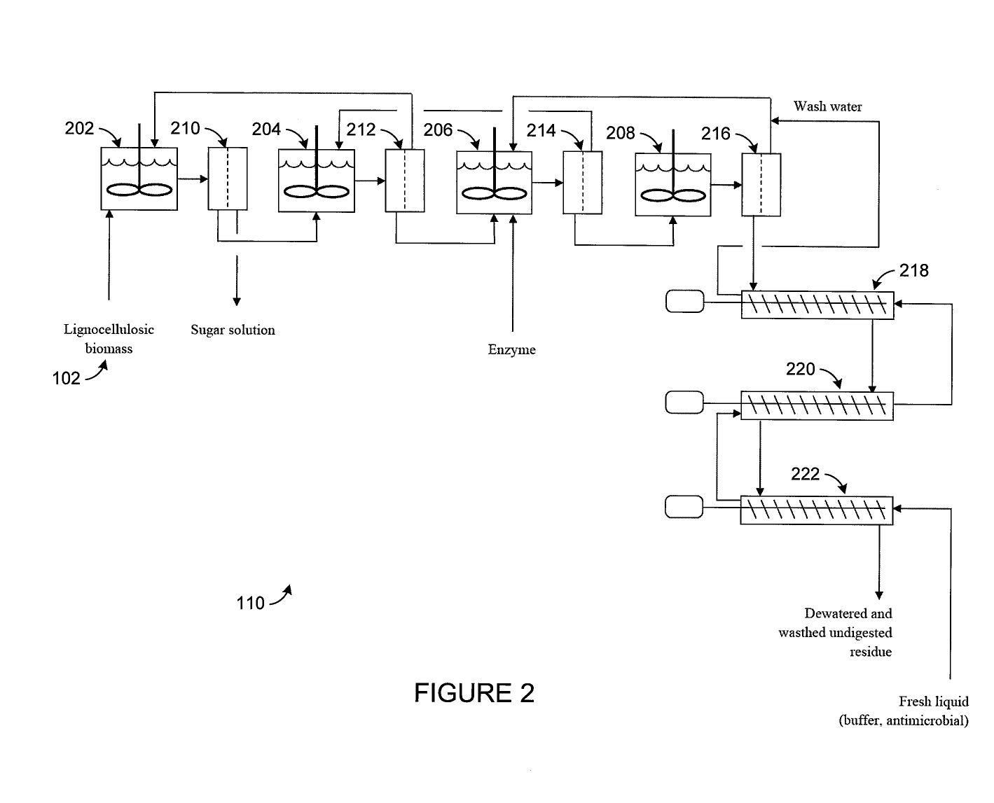

FIG. 2 illustrates an example option for achieving countercurrent saccharification according to this disclosure. More specifically, FIG. 2 illustrates one example embodiment for implementing the countercurrent saccharification stage 110 in the saccharification process 100 of FIG. 1.

As shown in FIG. 2, the countercurrent saccharification stage 110 includes various vessels 202-208, each of which is followed by a respective solids separator 210-216. Each vessel 202-208 denotes any suitable structure for holding one or more materials, such as a stirred tank. Each solids separator 210-216 includes any suitable structure for separating materials, such as a filter, centrifuge, or settler.

In some embodiments, enzyme is added to an intermediate vessel in the series of vessels 202-208. In this example, enzyme is added to the vessel 206. This allows the enzyme to flow in either direction (left-to-right or right-to-left) in the series of vessels 202-208.

Spent solids that exit the vessel 208 are countercurrently washed with fresh liquid (such as water containing buffer and antimicrobials) to recover interstitial sugars. Any suitable technique could be used to countercurrently wash spent solids. For example, the countercurrent wash system can employ screw presses or roller presses, such as those used in the sugar industry. Such presses employ a “hard squeeze” to remove interstitial water. Although use of the presses involves a small number of stages (such as two to four stages), the capital, energy, and maintenance costs are typically high. Alternatively, a series of screw conveyors 218-222 could be employed, which use a “gentle squeeze” to remove interstitial water. Although this approach may require the use of more stages (such as three to ten stages), screw conveyors typically require less capital, energy, and maintenance. In this example, there are three screw conveyors 218-222, although any number of screw conveyors could be used.

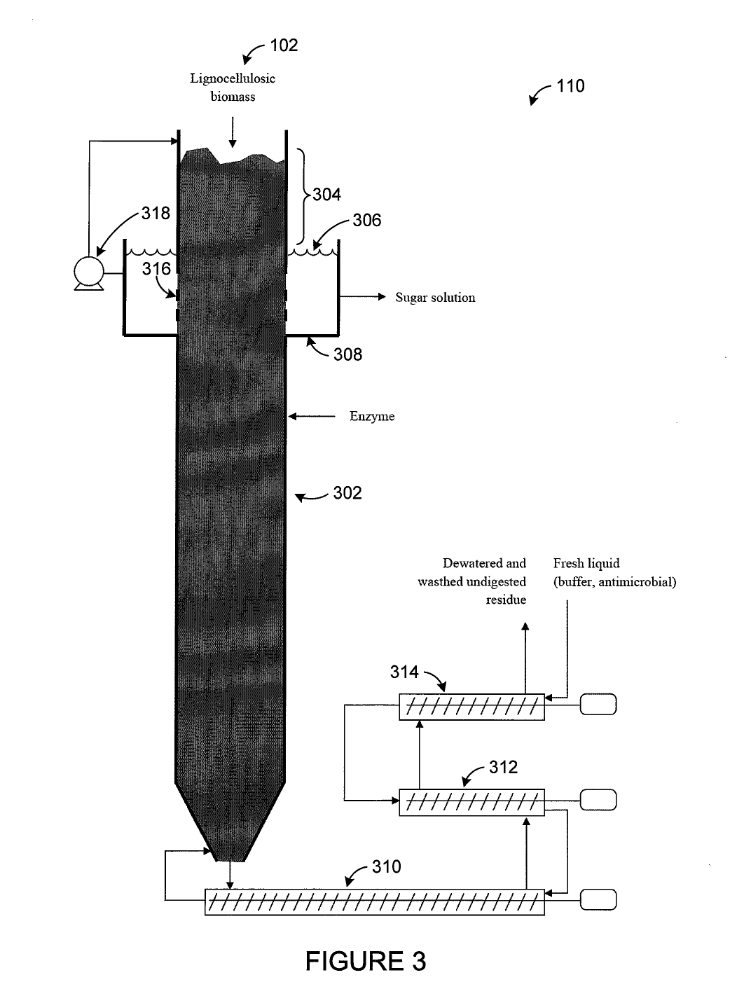

FIG. 3 illustrates an example option for achieving countercurrent flow in a packed column according to this disclosure. More specifically, FIG. 3 illustrates another example embodiment for implementing the countercurrent saccharification stage 110 in the saccharification process 100 of FIG. 1.

As shown in FIG. 3, lignocellulosic biomass 102 is added at the top of a column 302. The column 302 generally denotes an elongated structure in which biomass 102 flows from the top down and water flows from the bottom up in the column 302. To help ensure that the biomass 102 in the column 302 does not float in the water, a portion 304 of the biomass 102 is added above a level 306 of water within and surrounding the column 302. This adds weight to the biomass 102, keeping the column 302 well packed. In some embodiments, enzyme is added at an intermediate position in the column 302 between the top and bottom of the column 302.

To reduce or prevent the water from short-circuiting and exiting the bottom of the column 302, a valve (not shown) at the bottom of the column 302 can selectively pass solid but not liquid material. Examples of such a valve include one or more rotary lock hopper valves or, as shown in FIG. 3, one or more screw conveyors 310-314 with tightly packed solids. At the top of the column 302, water exits from a porous section 316 of the column's wall and is collected in one or more troughs 308. The water is circulated from the troughs 308 to the top of the pile by a pump 318, which allows saccharification to occur above the liquid level 306.

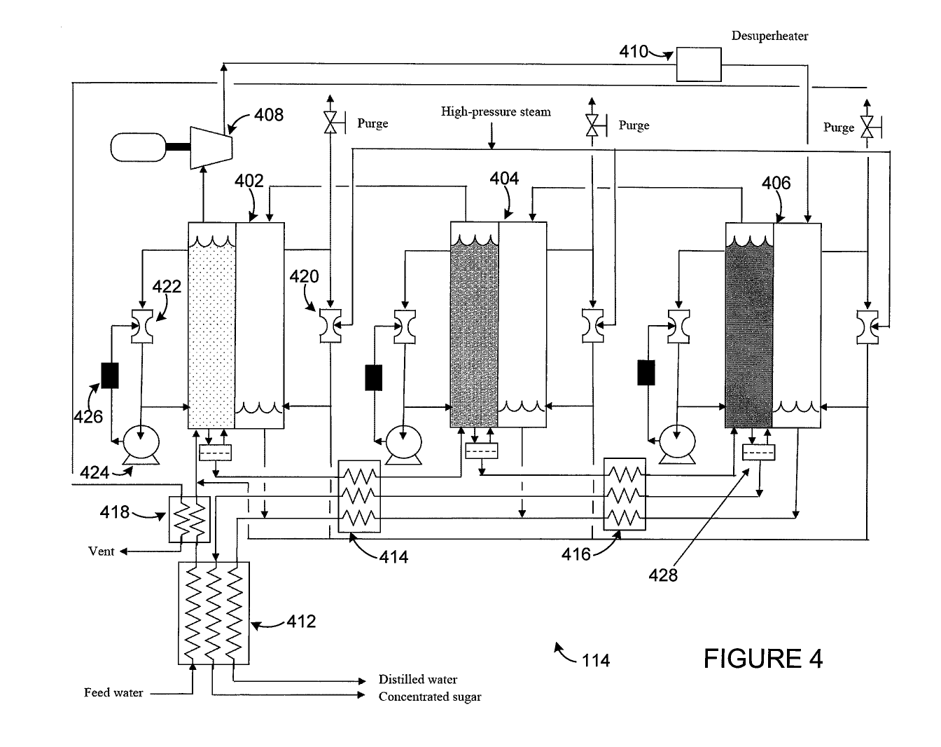

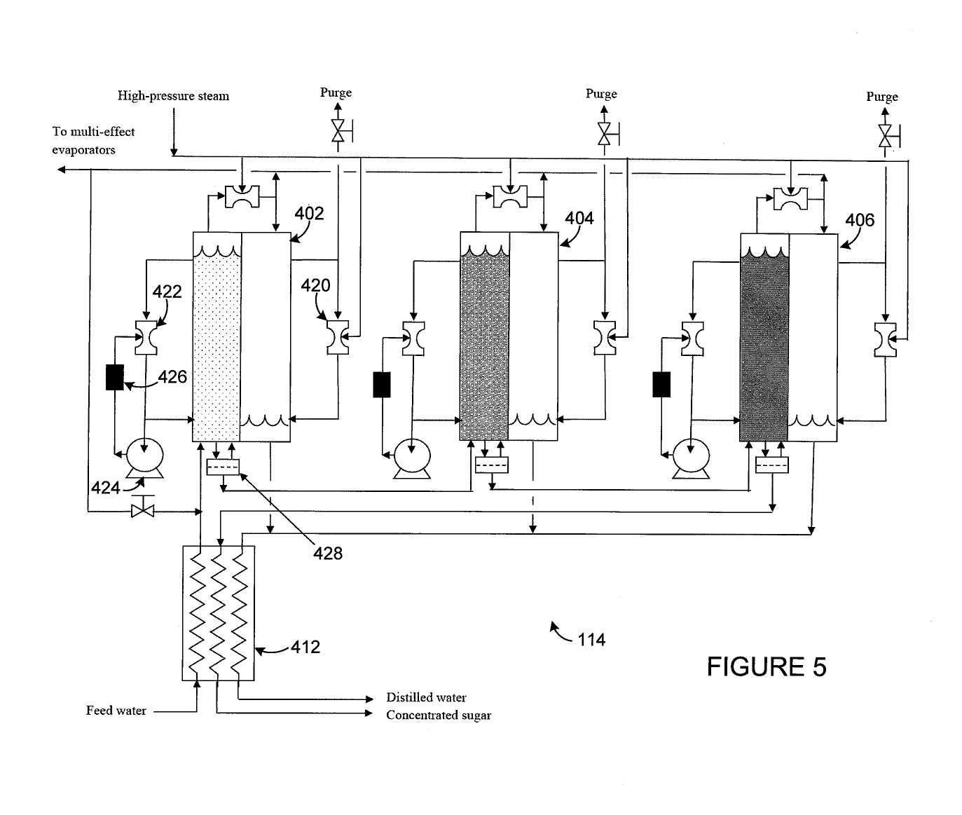

As noted above, the sugar concentrator stage 114 could implement vapor compression as a mechanism for concentrating the sugar solution from the countercurrent saccharification stage 110. FIGS. 4 and 5 illustrate example vapor-compression concentrator systems according to this disclosure. More specifically, FIG. 4 illustrates a vapor-compression concentrator system that uses a mechanical compressor, while FIG. 5 illustrates a vapor-compression concentrator system that uses jet ejectors.

In the example shown in FIG. 4, the sugar concentrator stage 114 uses three evaporator stages 402-406, although fewer or more evaporator stages could be employed. In this example, the evaporator stage 402 is at the lowest pressure, and the evaporator stage 406 is at the highest pressure. In the left portion of the evaporator stage 402, a vapor space above boiling water is connected to an inlet of a compressor 408. The work added to the compressor 408 causes discharged steam to be superheated. The superheat can be removed in a desuperheater 410, which can be accomplished by contacting the superheated steam with liquid water. When the liquid and vapor equilibrate, the steam becomes saturated (desuperheated). To facilitate heat transfer from the superheated steam to the liquid water, liquid water can be added as a fine mist. Alternatively, in a packed column, liquid water can countercurrently contact the superheated steam.

Saturated high-pressure steam that exits the desuperheater 410 enters the condensing side in the right portion of the evaporator stage 406. As this steam condenses, it evaporates water from the boiling side in the left portion of the evaporator stage 406, thereby producing steam that can be fed to the evaporator stage 404. In the evaporator stage 404, the steam condenses, which causes more steam to be produced on the boiling-water side. This steam then enters the evaporator stage 402, where it condenses and evaporates water from the boiling side. The water evaporated from the boiling side enters the compressor 408 as previously described.

To preheat the feeds to the evaporator stages 402-406, a sensible heat exchanger 412 can be employed, which exchanges thermal energy between (i) incoming feed water and (ii) discharged distilled water and concentrated brine. As shown in FIG. 4, the preheated feed water is fed to the evaporator stage 402. In a countercurrent series manner, a sugar solution exiting the evaporator stage 402 is directed to the evaporator stage 404, and a sugar solution exiting the evaporator stage 404 is directed to the evaporator stage 406. As the sugar solution flows from left to right, it becomes ever more concentrated. In the evaporator stage 402 (at the lowest sugar concentration), the pressure ratio between the condensing steam and boiling water is minimal. In the evaporator stage 406 (at the highest sugar concentration), the pressure ratio between the condensing steam and boiling water is maximal.

In other embodiments, in a co-current series manner, preheated feed water could be added to the evaporator stage 406. In this arrangement, as the sugar solution flows from right to left, it becomes ever more concentrated. In the evaporator stage 406 (at the lowest sugar concentration), the pressure ratio between the condensing steam and boiling water is minimal. In the evaporator stage 402 (at the highest sugar concentration), the pressure ratio between the condensing steam and boiling water is maximal.

In still other embodiments, in a parallel manner, preheated feed water could be divided into three portions and added to each of the evaporator stages 402-406. In these embodiments, each evaporator stage 402-406 has the maximum sugar concentration. As a result, the pressure ratio between the condensing steam and boiling water is maximal in each evaporator stage 402-406, which may adversely affect energy efficiency because the compressor 408 has the maximum compression ratio.

Regardless of the flow arrangement, each evaporator stage 402-406 can operate at a different temperature. Therefore, to conserve energy, sensible heat exchangers 414-416 can be employed between adjacent pairs of evaporator stages 402-406.

Because non-condensable gases can be present in the feed water, the non-condensable gases can be purged from the system. For example, purged steam can include mostly steam with small amounts of non-condensables. The purged stream could simply be vented to the atmosphere, although this wastes the energy in the steam. Alternatively, as shown in FIG. 4, the purged stream can be sent to a heat exchanger 418, which helps preheat the incoming feed water.

In each of the evaporator stages 402-406, the steam-side (right-side) heat transfer coefficient improves by inducing a circulating flow, which can be accomplished in each evaporator stage 402-406 using a jet ejector 420 driven by high-pressure steam. A portion of this circulating flow can be bled and fed directly into the incoming feed, thereby assisting with preheating. Also, in each evaporator stage 402-406, the liquid-side (left-side) heat transfer coefficient improves by circulating liquid, which can be accomplished using a jet ejector 422 powered by a pump 424.

As sugar concentrates, there is the potential for fouling as salts attach to various heat exchanger surfaces. To help reduce or prevent this, a salt nucleation promoter 426 can be incorporated into each circulating flow. The salt nucleation promoter 426 could, for example, represent a COLLOID-A-TRON produced by FLUID DYNAMICS. The salt nucleation promoter 426 encourages salts to preferentially precipitate in the bulk liquid rather than on solid surfaces and thus helps to avoid fouling.

To promote vapor nucleation in the circulating liquid of each evaporator stage 402-406, “boiling chips” (such as TEFLON boiling chips sold by CR SCIENTIFIC) can be added. A further advantage of introducing boiling chips is that they abrade against heat exchanger surfaces and therefore help to remove scale. If boiling chips are employed, a separator 428 (such as a filter) can be used with each evaporator stage 402-406 to retain the boiling chips within that evaporator stage.

In the example shown in FIG. 5, the sugar concentrator stage 114 again uses three evaporator stages 402-406, although fewer or more evaporator stages could be employed. The heat exchanger 412 is employed to preheat the feeds to the evaporator stages 402-406. A circulating flow can be accomplished in each evaporator stage 402-406 using a jet ejector 420 driven by high-pressure steam. Also, a circulating liquid can be accomplished in each evaporator stage 402-406 using a jet ejector 422 powered by a pump 424. Salt nucleation promoters 426 and separators 428 can be used with the evaporator stages 402-406.

In this example, the jet ejectors 420-422 replace the mechanical compressor 408. Each evaporator stage 402-406 has its own jet ejectors 420-422, so each evaporator stage 402-406 can be operated at the same temperature and thus eliminate the need for the heat exchangers 414-416 between the evaporator stages 402-406. In FIG. 5, purged non-condensables are vented directly to the atmosphere, although the purged steam could be directed to the heat exchanger 418 that preheats the feed water (as shown in FIG. 4 but omitted from FIG. 5).

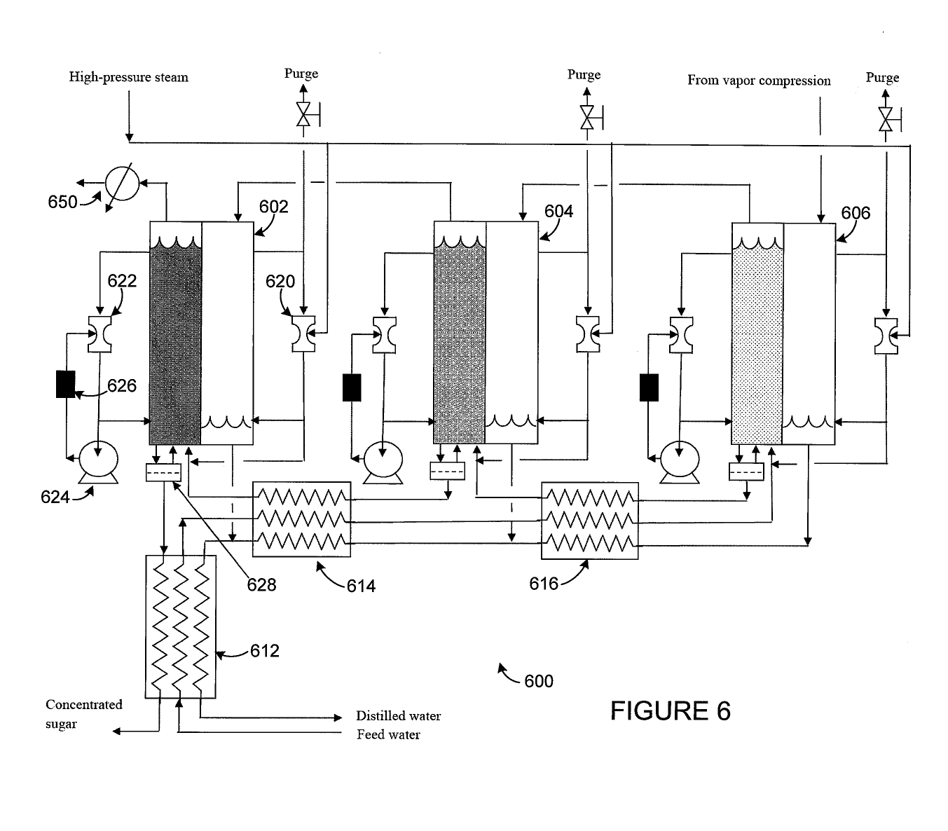

The steam that powers the jet ejectors 420-422 can also be purged from the system. By operating the evaporator stages 402-406 at high temperatures, the purged steam can actually be sent to multi-effect evaporators (shown in FIG. 6) to concentrate additional feed water. Alternatively, if a dewatering system is employed in a chemical plant, the purged steam can be used for other purposes, such as distillation.

FIG. 6 illustrates an example multi-effect evaporator system 600 according to this disclosure. The multi-effect evaporator system 600 can be used as part of the sugar concentrator stage 114 and used in conjunction with the vapor-compression concentrator system shown in FIG. 5. As shown in FIG. 6, the evaporator system 600 includes evaporator stages 602-606, heat exchangers 612-616, jet ejectors 620-622, pumps 624, salt nucleation promoters 626, and separators 628.

High-pressure steam from the vapor-compression system enters the evaporator stage 606, which operates at the highest pressure. When this steam condenses, it transfers heat to the boiling liquid, where additional steam is produced but at lower pressure. This steam is fed to the evaporator stage 604, where the same process occurs. The steam produced in the evaporator stage 604 is sent to the evaporator stage 602. In FIG. 6, three multi-effect evaporator stages 602-606 are shown, although any number of multi-effect evaporator stages can be used (including a large number of stages). As the steam flows from right to left in FIG. 6, the temperature lowers. Ultimately, the temperature is too low to be useful, and the steam produced from the last evaporator stage 602 is condensed in a condenser 650 that rejects the heat to cooling water or air. The high-pressure steam used in the jet ejector 620 that circulates steam on the condensing side of the evaporator stage 606 can be vented. It could also be added directly to the incoming feed to preheat it to saturation conditions.

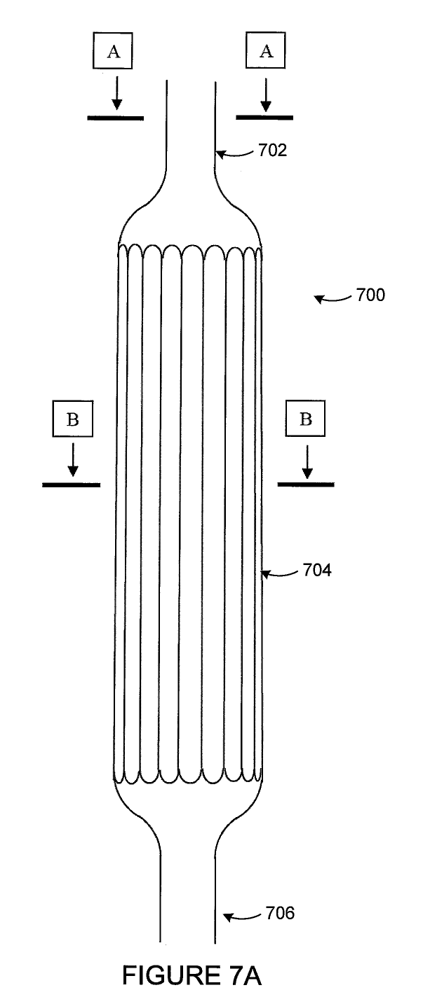

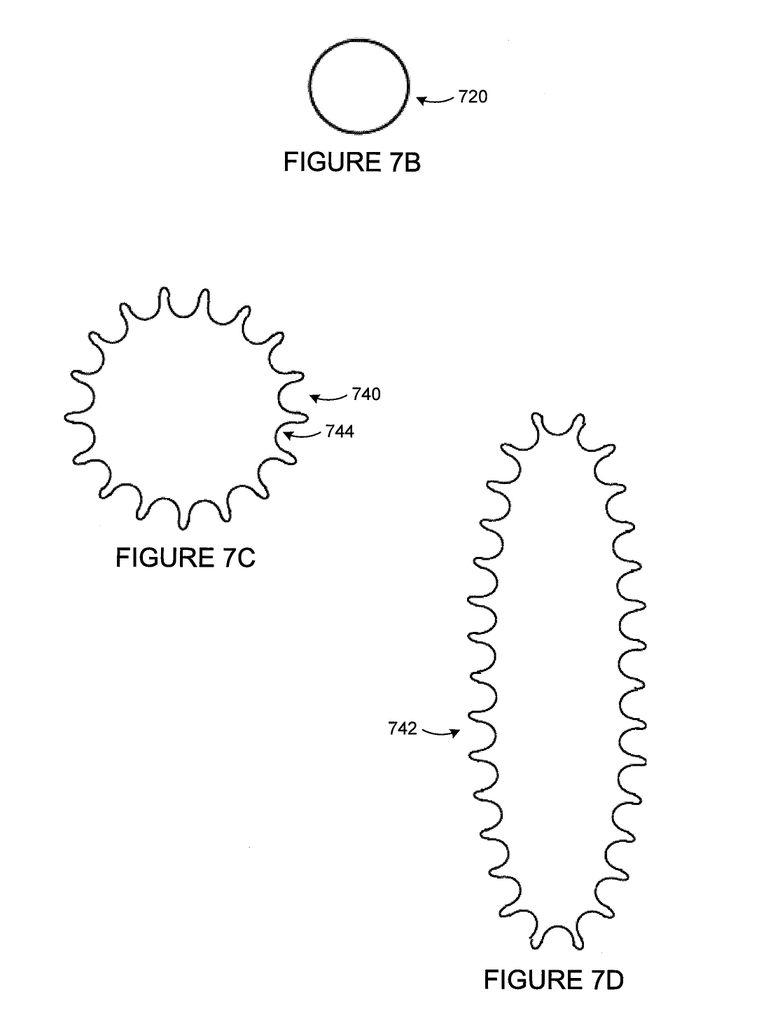

As described above, at least one component in the saccharification process 100 includes a heat exchanger. FIGS. 7A through 7D illustrate an example individual heat exchanger tube 700 according to this disclosure. Multiple heat exchanger tubes 700 can be used to form the various heat exchangers in the saccharification process 100, although any other suitable heat exchanger tubes could also be used.

As shown in FIG. 7A, the heat exchanger tube 700 includes an inlet section 702, a heat exchange section 704, and an outlet section 706. One example cross-sectional shape 720 of the inlet section 702 is shown in FIG. 7B, where the inlet section 702 has a substantially circular cross-sectional shape. The outlet section 706 could have the same or similar cross-sectional shape or a different cross-sectional shape. Example substantially circular and oval cross-sectional shapes 740-742 of the heat exchange section 704 are shown in FIGS. 7C and 7D, where vertical grooves 744 are formed within the heat exchange section 704. The cross-sectional shapes 720, 740-742 in FIGS. 7B through 7D are examples only, and the heat exchanger tube 700 could have any other suitable cross-sectional shape(s) in any portion(s) of the heat exchanger tube 700.

The vertical grooves 744 in the heat exchanger tube 700 could be formed in any suitable manner. For example, using hydroforming, the vertical grooves 744 can be created by placing a cylindrical tube in a mold and increasing the internal pressure beyond the yield point. Experimental data indicates that vertical grooves 744 have superior heat transfer coefficients, which presumably occurs because liquid droplets that form at the tops of the grooves 744 flow downward in the vertical channels and clear liquid adhering to the surface at the lower portions of the tube 700.

In some embodiments, the tube 700 is made from a high thermal conductivity material, such as copper. Optionally, the tube's interior can be sand-blasted or otherwise processed to create nucleation sites. The entire tube 700 can be coated with nickel-TEFLON or other material to promote dropwise condensation and to resist fouling. Alternatively, a nickel or other coating can incorporate carbon nanotubes, which are also hydrophobic. Advantageously, carbon nanotubes have a high thermal conductivity, unlike TEFLON.

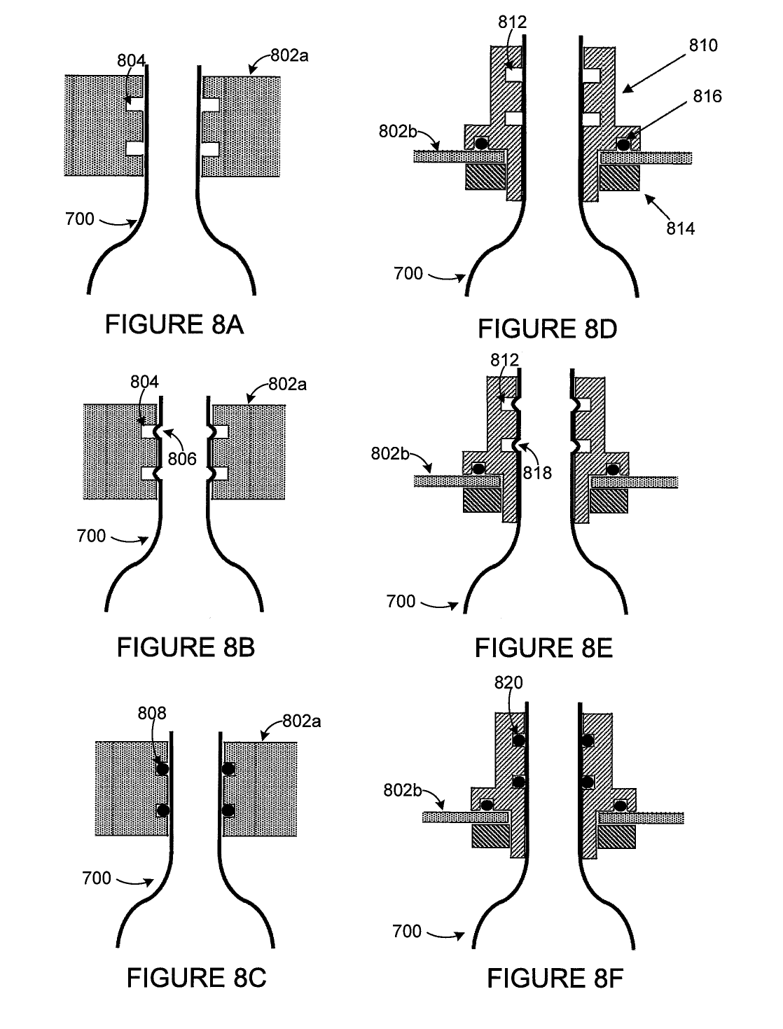

FIGS. 8A through 8F illustrate example methods for joining a heat exchanger tube 700 to a tube sheet 802a or 802b according to this disclosure. FIGS. 8A through 8C show methods where the tube sheet 802a is thick. As shown in FIG. 8A, grooves 804 are formed inside a hole in the tube sheet 802a, and the tube 700 is placed through the hole in the tube sheet 802a. As shown in FIG. 8B, the tube 700 is swaged to form small indentations 806 that fit within the grooves 804, thereby forming a seal between the tube 700 and the tube sheet 802a. In some embodiments, the seal can be formed using HYDROSWAGE technology from HASKEL. As shown in FIG. 8C, sealing can alternatively be accomplished using O-rings or other seals 808 within the grooves 804.

FIGS. 8D through 8F show methods where the tube sheet 802b is thin. As shown in FIG. 8C, the tube 700 and a fitting 810 are installed into the tube sheet 802b. The fitting 810 includes grooves 812, and a nut 814 can be used to secure the fitting 810 to the tube sheet 802b. Also, an O-ring or other seal 816 can be used between the fitting 810 and the tube sheet 802b. As shown in FIG. 8E, the tube 700 is swaged to form small indentations 818 that fit within the grooves 812 of the fitting 810, thereby forming a seal between the tube 700 and the tube sheet 802b. In some embodiments, the seal can be formed using HYDROSWAGE technology from HASKEL. As shown in FIG. 8F, sealing can alternatively be accomplished using O-rings or other seals 820 within the grooves 812.

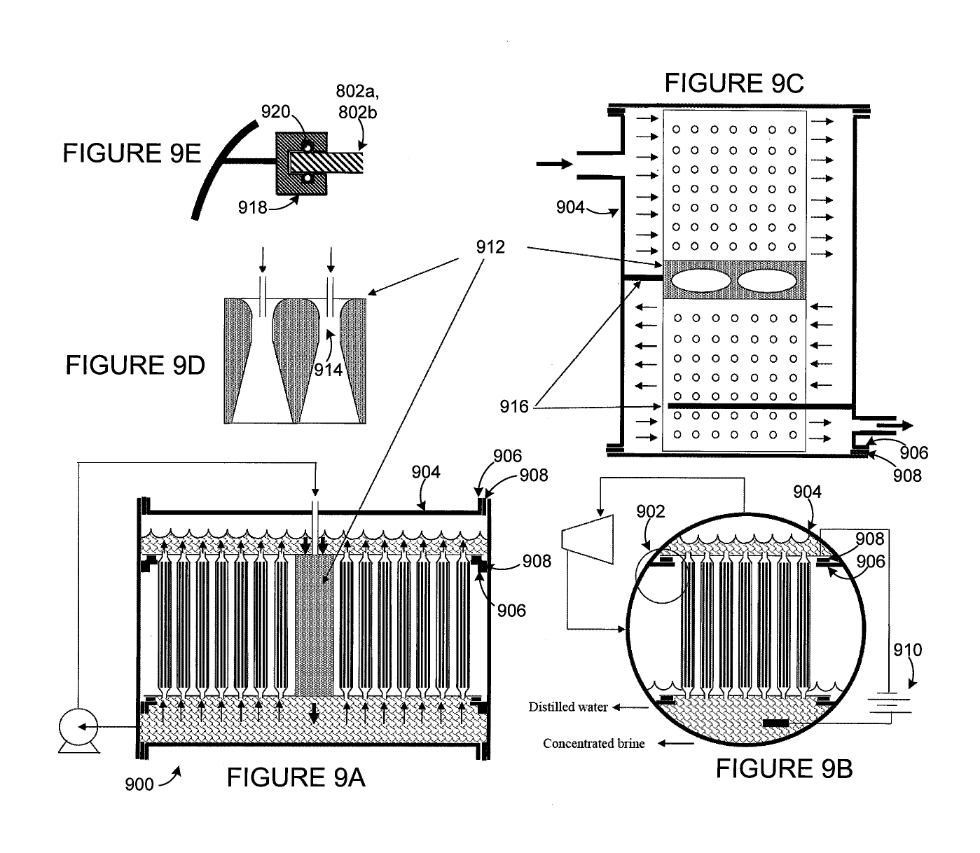

FIGS. 9A through 9E illustrate an example assembled heat exchanger 900 that employs multiple heat exchanger tubes according to this disclosure. The heat exchanger 900 can employ the heat exchanger tubes 700 of FIG. 7 joined using one of the techniques shown in FIGS. 8A through 8F, although other heat exchangers could also be used. FIG. 9A shows a side view of the heat exchanger 900, FIG. 9B shows a front view of the heat exchanger 900, and FIG. 9C shows a top view of the heat exchanger 900. Also, FIG. 9D illustrates a close-up view of jet ejectors used in the heat exchanger 900, and FIG. 9E illustrates a close-up view of an alternative sealing technique that could be used in the circled area 902 of FIG. 9B.

As shown in FIGS. 9A through 9C, a shell 904 of the heat exchanger 900 has various tabs 906 on its interior wall(s) that allow the tube sheets 802a or 802b to be sealed using gaskets 908. Each tube's exterior is the steam condenser, whereas each tube's interior is the boiler. Sugar water from a lower portion of the heat exchanger 900 flows upward through each tube's interior. When it emerges from the top, its vapors disentrain and are sent to a compressor inlet. Compressed vapor is directed to each tube's exterior, where the vapor condenses and is collected as distilled water.

Some high thermal conductivity metals (such as copper) can be corroded in a salty environment. In some embodiments, corrosion can be reduced or prevented by using galvanic protection, such as by imposing an impressed current (shown here as being provided by a voltage source 910) or using a sacrificial electrode (not shown). In other embodiments, no galvanic protection may be needed if the tubes, tube sheets, and fittings are all made from the same alloy and the assembly is electrically isolated from dissimilar metals.

In FIGS. 9A, 9C, and 9D, jet ejectors 912 are shown as being incorporated into the heat exchanger 900. Using a pump, liquid can be drawn from the bottom and pumped into nozzles located at the throats 914 of the jet ejectors 912. The jet ejectors 912 force water from the top portion of the heat exchanger 900 into the bottom portion of the heat exchanger 900. The liquid returns to the top through the tubes' interiors. The jet ejectors 912 thereby impose forced circulation, which improves heat transfer.

FIG. 9C shows various baffles 914 that provide a substantially uniform velocity as steam flows past the tubes. The baffles' spacing reduces as the steam flows to an exit. This flow pattern also forces non-condensables to accumulate at the exit, where they can be purged.

FIG. 9E shows an alternative method for sealing a tube sheet 802a or 802b to the shell 904 of the heat exchanger 900. A C-shaped extrusion 918 is attached to the inside shell wall. The inside of the extrusion 918 has one or more grooves 920 that allow an inflatable linear seal to be inserted. During assembly, when the tube sheet 802a or 802b is slid into the C-shaped extrusion 918, the linear seals are not inflated. Once the tube sheet 802a or 802b is inserted, the linear seals are inflated. One advantage of this sealing system is that it allows heat exchangers to be rapidly installed or replaced without the difficulty of accessing bolts, as would be needed in a conventional gasket seal.

In FIGS. 9A through 9E, the shell's axis and the tubes' axes are at right angles, which is a nontraditional arrangement. In other embodiments, the shell's axis and tubes' axes are parallel, which is the traditional arrangement for shell-and-tube heat exchangers. Any other suitable orientations of the shell's axis and the tubes' axes could also be used.

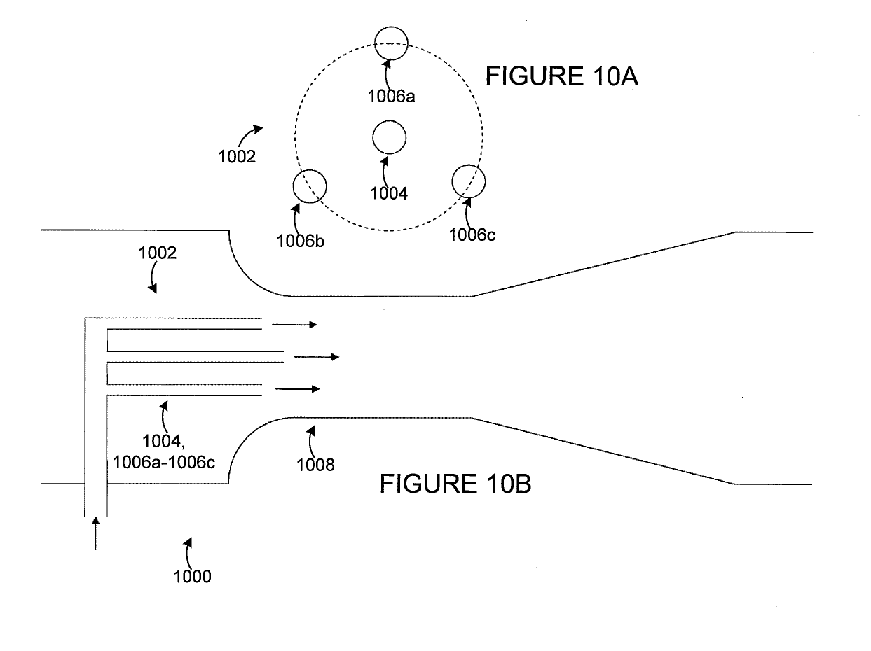

A properly designed jet ejector improves the energy efficiency of a vapor-compression system (such as the ones shown in FIGS. 4 and 5). FIGS. 10 through 12 illustrate example jet ejector designs 1000, 1100, 1200 according to this disclosure. In each jet ejector design, a nozzle 1002, 1102, 1202 includes a central tube 1004, 1104, 1204 surrounded by multiple “satellite” tubes 1006a-1006c, 1106a-1106c, 1206a-1206c. While three satellite tubes are shown here, more or fewer satellite tubes may be used.

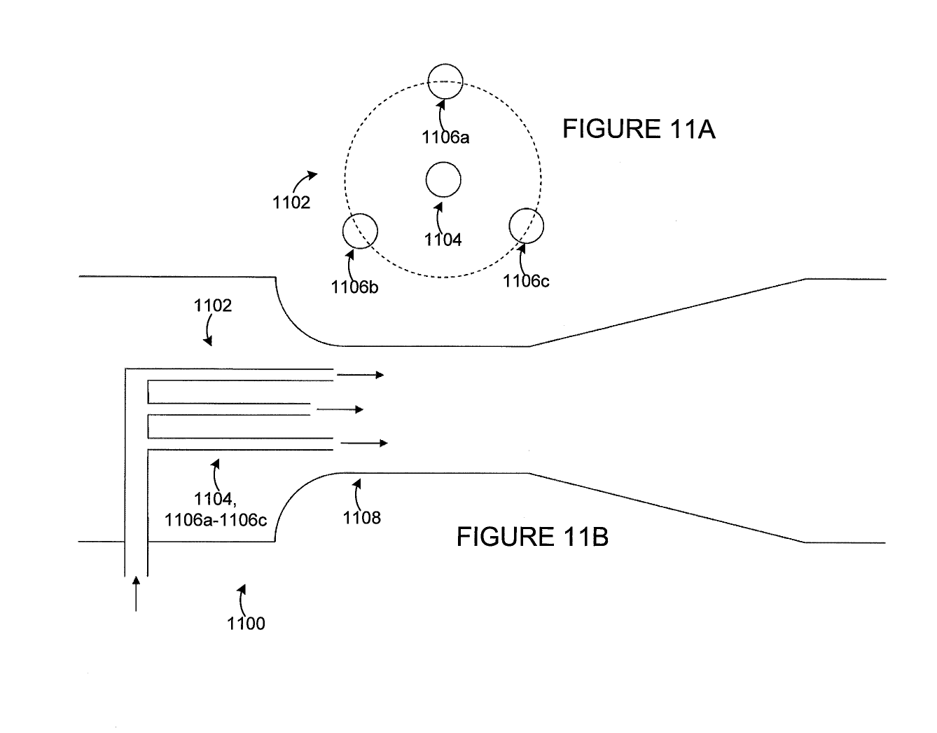

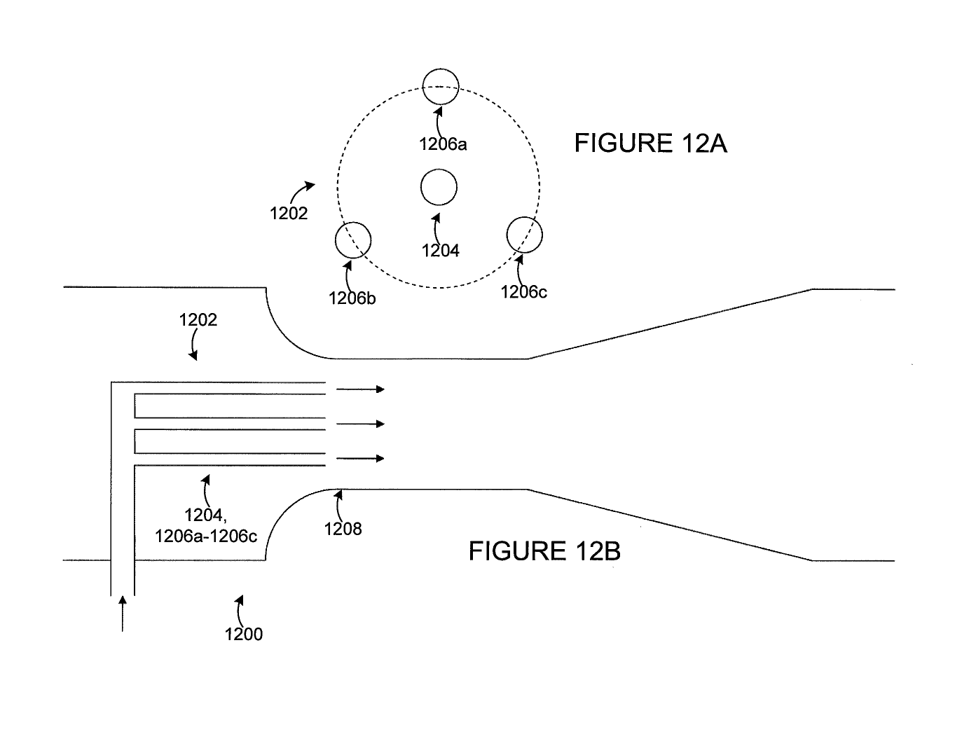

The tips of the tubes 1004 and 1006a-1006c, 1104 and 1106a-1106c, 1204 and 1206a-1206c can be staggered or aligned. Various computer models indicate that the best arrangement is for the central tube's outlet to be located at the entrance to the mixing tube 1008 (the constant-diameter tube at the center) and the satellite tubes 1006a-1006c are backed off slightly as shown in FIG. 10. It is possible to reverse this with the exits of the satellite tubes 1106a-1106c located at the entrance to the mixing tube 1108 and the central tube 1104 backed off slightly as shown in FIG. 11. Also, all tube tips can all be aligned as shown in FIG. 12. Any other suitable arrangement could also be used.

In principle, the central and satellite tubes can be inserted more deeply into the mixing tubes 1008, 1108, 1208, or they can be moved leftward away from the entrances to the mixing tubes 1008, 1108, 1208. Various computer models indicate that these may be non-optimal arrangements, but they are possible alternative embodiments.

The following represents experimental data associated with particular implementations of the saccharification process 100. This experimental data is specific to the particular implementations of the saccharification process 100 and does not limit the scope of this invention to these particular implementations.

Table 1 shows an example composition of alpha cellulose used to test the countercurrent saccharification process. In batch saccharifications, the initial solids concentration was 100 g/L (10 g solids/90 g water). In continuous saccharifications, 10 g solids were loaded into the saccharification train for every 90 g of water. Each saccharification train included eight 1-L centrifuge bottles. Enzyme was added to bottle #4 (B4). After centrifuging the bottles, excess liquid was passed in one direction, and excess solids were passed in the opposite direction. Sugar solution was harvested from bottle #1 (B1), and spent solids were discarded from bottle #8 (B8). In each bottle, the biomass concentration was maintained as high as possible with very little free liquid.

TABLE 1

| Component | Composition (%) |
|---|---|
| Glucan | 77.4 |
| Xylan | 14.1 |
| Galactan | 0.6 |
| Extractives | 0.7 |
| Other | 7.2 |
| Total | 100 |

Table 2 shows the enzyme loadings used in both the batch and continuous saccharifications. Saccharifications were performed at 50° C. and at a pH of 4.8 using Novozyme Cellic CTec 2.

TABLE 2

| | Enzyme Loading | Enzyme Loading |
|---|---|---|
| | (mg protein/g dry solid) | (mg protein/g glucan) |
| Batch | | |
| A | 1 | 1.29 |
| B | 2 | 2.58 |
| C | 5 | 6.46 |
| D | 10 | 12.92 |
| E | 25 | 32.30 |
| Continuous | | |
| Train 1 | 2 | 2.58 |
| Train 2 | 5 | 6.46 |

Table 3 describes the properties of the enzyme. The protein content was assessed using a Pierce bicinchoninic acid (BCA) protein assay, and filter paper activity was assessed by the standard National Renewable Energy Laboratory (NREL) method (NREL/TP-510-42629).

TABLE 3

| | | Protein | Filter Paper | Specific |
|---|---|---|---|---|
| | | Conc. | Activity | Activity |
| Commercial Product | Activity | (mg/mL) | (FPU/mL) | (FPU/g) |
| Novozyme Cellic CTec 2 | cellulase | 294 ± 32 | 225 ± 20 | 765 |

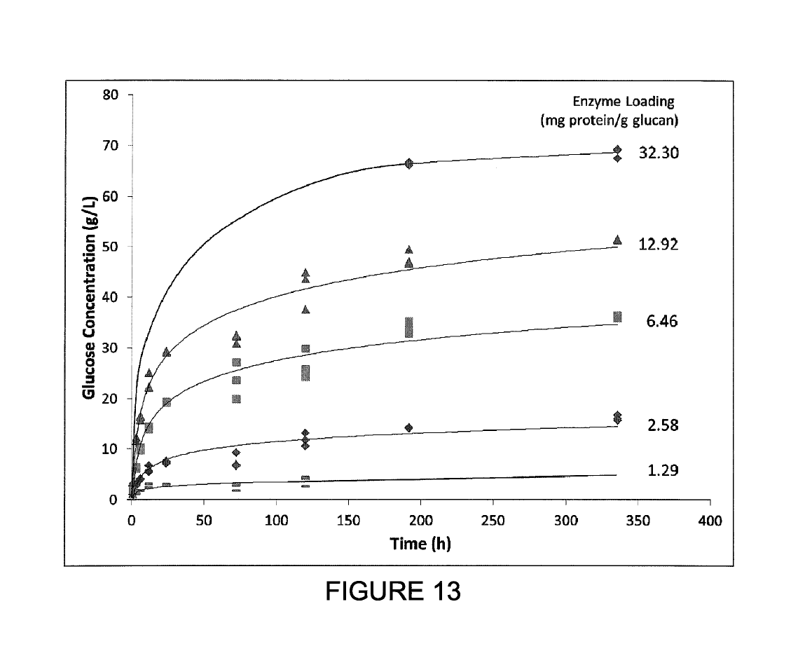

FIG. 13 illustrates an example glucose concentration as a function of time according to this disclosure. The reaction is complete in 336 hours. For the various enzyme loadings, Table 4 shows the conversion of glucan and xylan at 336 hours.

TABLE 4

| | | | | |
|---|---|---|---|---|
| Enzyme loading | 2 | 5 | 10 | 25 |
| (mg protein/g dry solids) | | | | |
| Enzyme loading | 2.58 | 6.46 | 12.92 | 32.30 |
| (mg protein/g glucan) | | | | |

Glucose

| | | | | |
|---|---|---|---|---|
| Replicate 1 (g/L) | 15.65 | 35.74 | 51.29 | 67.47 |
| Replicate 2 (g/L) | 15.94 | 35.92 | 51.35 | 69.23 |
| Replicate 3 (g/L) | 16.71 | 36.39 | 51.49 | 69.10 |
| Average (g/L) | 16.10 | 36.02 | 51.37 | 68.60 |
| Max potential glucose | 86.00 | 86.00 | 86.00 | 86.00 |
| (g/L) | | | | |
| Conversion (%) | 18.72 | 41.88 | 59.74 | 79.77 |

Xylose

| | | | | |
|---|---|---|---|---|
| Replicate 1 (g/L) | 2.55 | 4.44 | 5.80 | 9.26 |
| Replicate 2 (g/L) | 2.56 | 4.78 | 5.83 | 9.55 |
| Replicate 3 (g/L) | 2.95 | 4.48 | 5.99 | 9.46 |
| Average (g/L) | 2.68 | 4.57 | 5.87 | 9.42 |
| Max potential xylose (g/L) | 16.02 | 16.02 | 16.02 | 16.02 |
| Conversion (%) | 16.76 | 28.52 | 36.66 | 58.82 |

An example of the calculations performed to generate the values in Table 4 includes the following.

$$Max \cdot potential glucose = \frac{100 g solids}{L} \times \frac{0.774 g glucan}{g solids} \times \frac{1.1111 g glucose}{g glucan} = 86.00 \frac{g glucose}{L}$$

$$Max \cdot potential xylose = \frac{100 g solids}{L} \times \frac{0.141 g xylan}{g solids} \times \frac{1.136 g xylose}{g xylan} = 16.02 \frac{g xylose}{L}$$

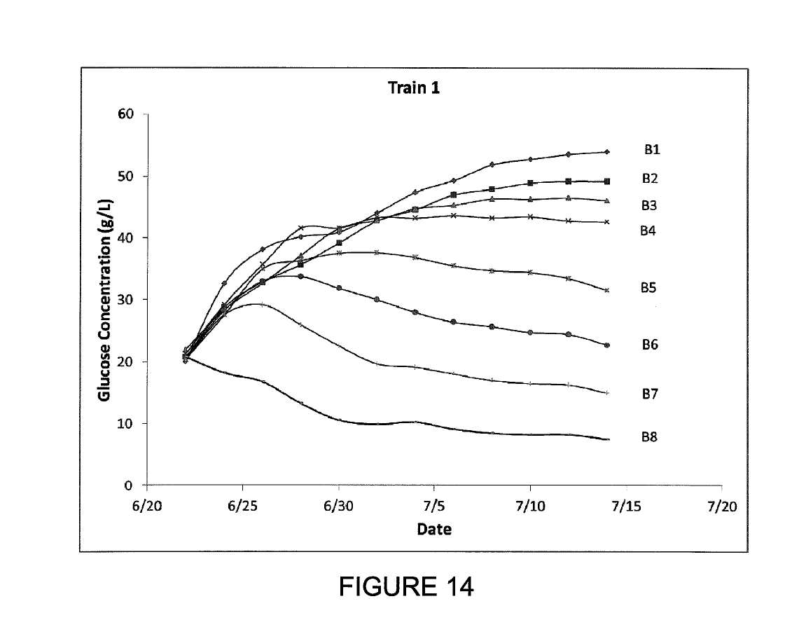

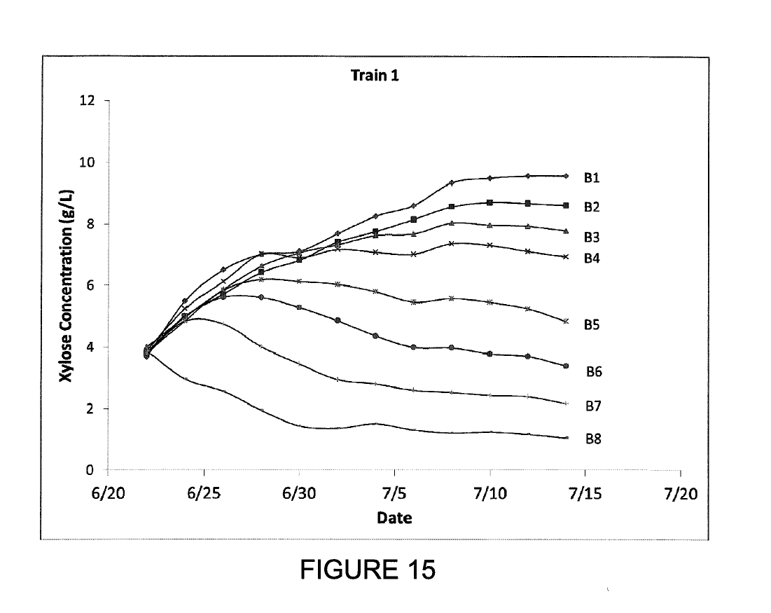

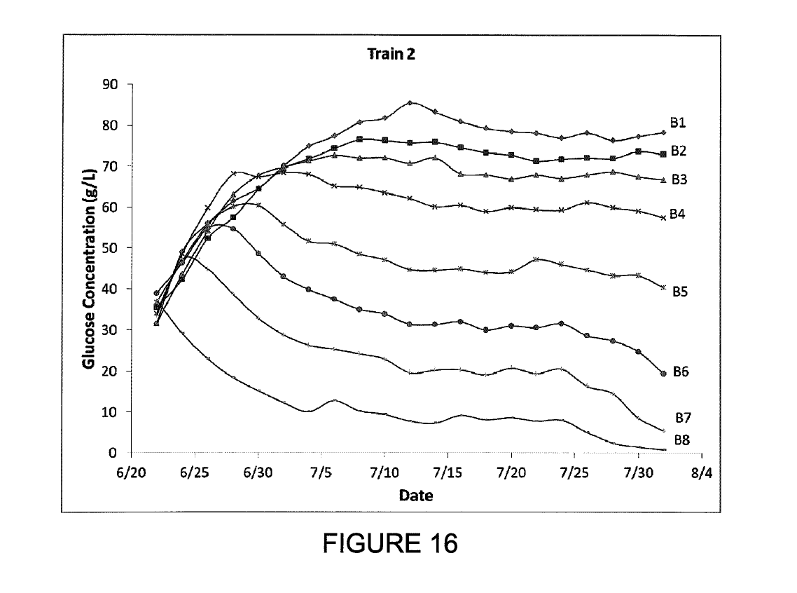

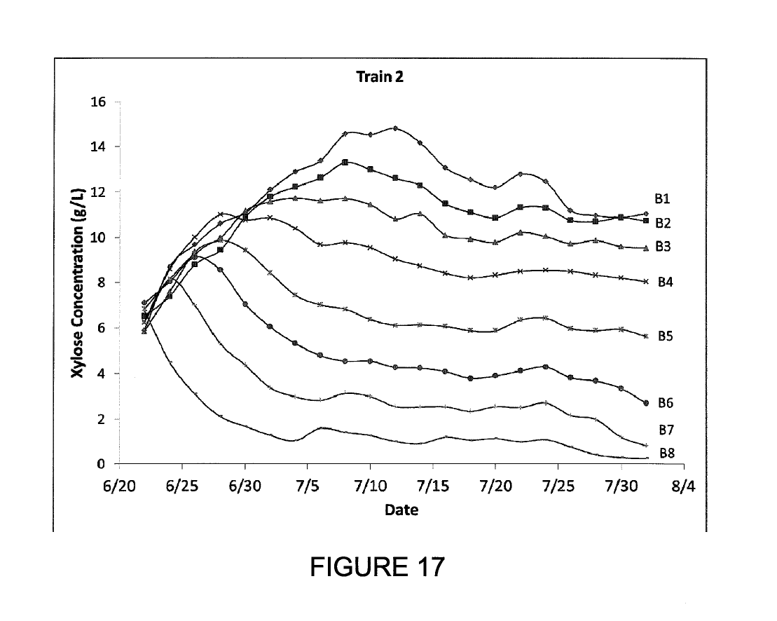

FIGS. 14 and 15 illustrate example glucose and xylose concentrations in each bottle of a first train of the saccharification process as a function of time according to this disclosure. FIGS. 16 and 17 illustrate example glucose and xylose concentrations in each bottle of a second train of the saccharification process as a function of time according to this disclosure.

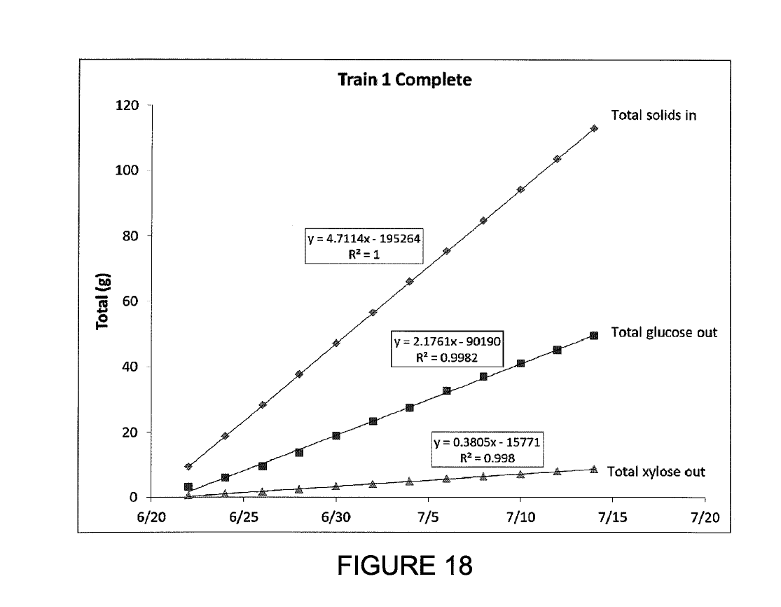

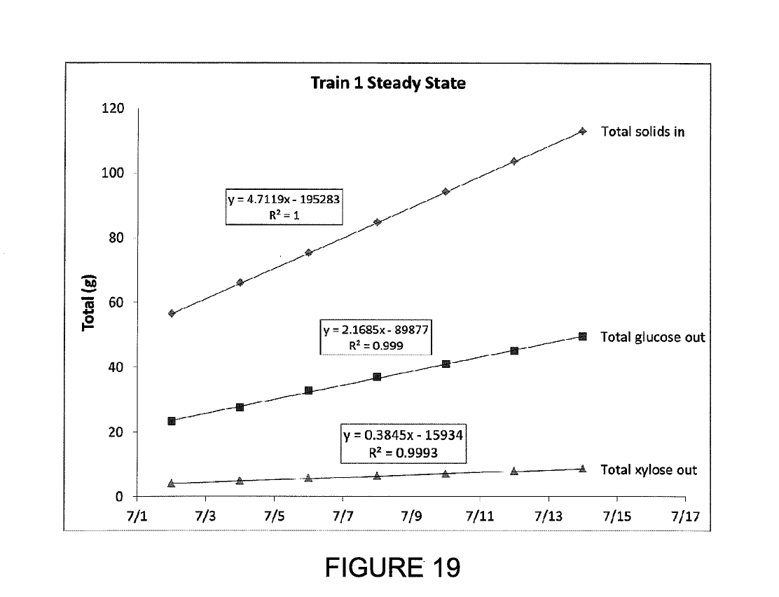

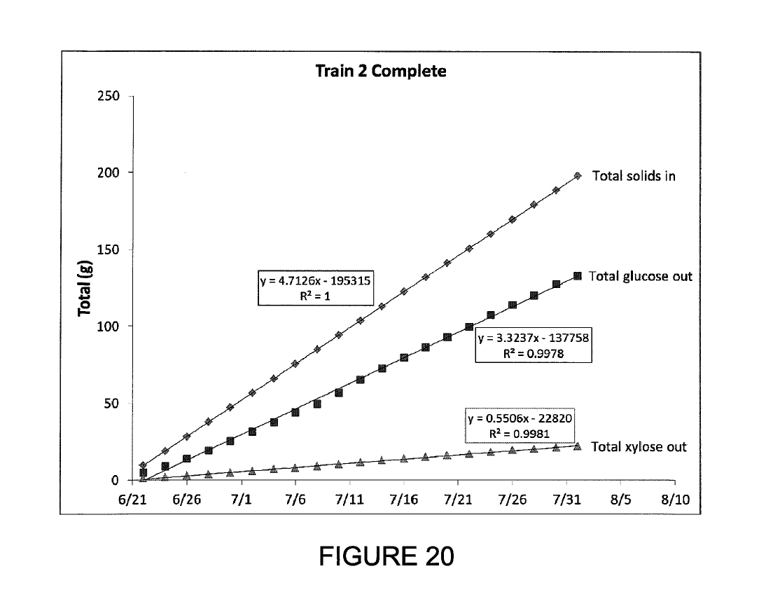

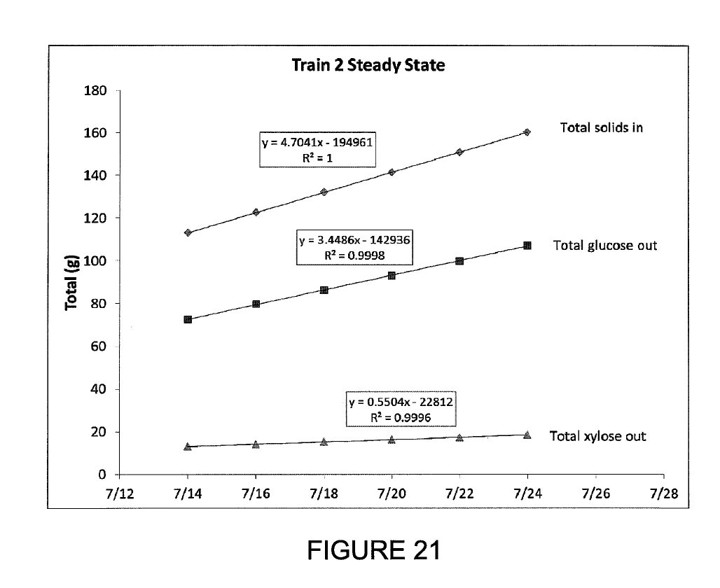

FIG. 18 illustrates example total solids in, total glucose out, and total xylose out for an entire experiment in the first train of the saccharification process according to this disclosure. FIG. 19 illustrates example total solids in, total glucose out, and total xylose out for a steady-state portion of the experiment in the first train of the saccharification process according to this disclosure. FIG. 20 illustrates example total solids in, total glucose out, and total xylose out for an entire experiment in the second train of the saccharification process according to this disclosure. FIG. 21 illustrates example total solids in, total glucose out, and total xylose out for a steady-state portion of the experiment in the second train of the saccharification process according to this disclosure. Note that total sugar output includes product liquid exiting bottle B 1, entrained liquid exiting bottle B8, and samples taken from each bottle.

Table 5 shows that the glucan conversion is 53.7% and 85.3% for Trains 1 and 2, respectively. The xylan conversion is 50.9% and 73.0% for Trains 1 and 2, respectively.

TABLE 5

| | Train 1 | Train 2 |
|---|---|---|
| Enzyme loading (mg protein/g dry solids) | 2 | 5 |
| Enzyme loading (mg protein/g glucan) | 2.58 | 6.46 |

Glucose

| | | |
|---|---|---|
| Slope (g glucose/day) | 2.169 | 3.449 |
| Maximum potential slope (g glucose/day) | 4.042 | 4.045 |
| Conversion (%) | 53.7 | 85.3 |

Xylose

| | | |
|---|---|---|
| Slope (g xylose/day) | 0.3845 | 0.5504 |
| Maximum potential slope (g xylose/day) | 0.7547 | 0.7535 |
| Conversion (%) | 50.9 | 73.0 |

An example of the calculations performed to generate the values in Table 5 includes the following.

$$\begin{matrix}Max \cdot potential glucose = \frac{4.7119 g solids}{d} \times \frac{0.774 g glucan}{g solids} \times \frac{1.1111 g glucose}{g glucan} = 4.042 \frac{g glucose}{d} \\ Max \cdot potential xylose = \frac{4.7041 g solids}{d} \times \frac{0.141 g xylan}{g solids} \times \frac{1.136 g xylose}{g xylan} = 0.7547 \frac{g xylose}{d} & Train 1 \\ Max \cdot potential glucose = \frac{4.7041 g solids}{d} \times \frac{0.774 g glucan}{g solids} \times \frac{1.1111 g glucose}{g glucan} = 4.045 \frac{g glucose}{d} \\ Max \cdot potential xylose = \frac{4.7041 g solids}{d} \times \frac{0.141 g xylan}{g solids} \times \frac{1.136 g xylose}{g xylan} = 0.7535 \frac{g glucose}{d} & Train 2\end{matrix}$$

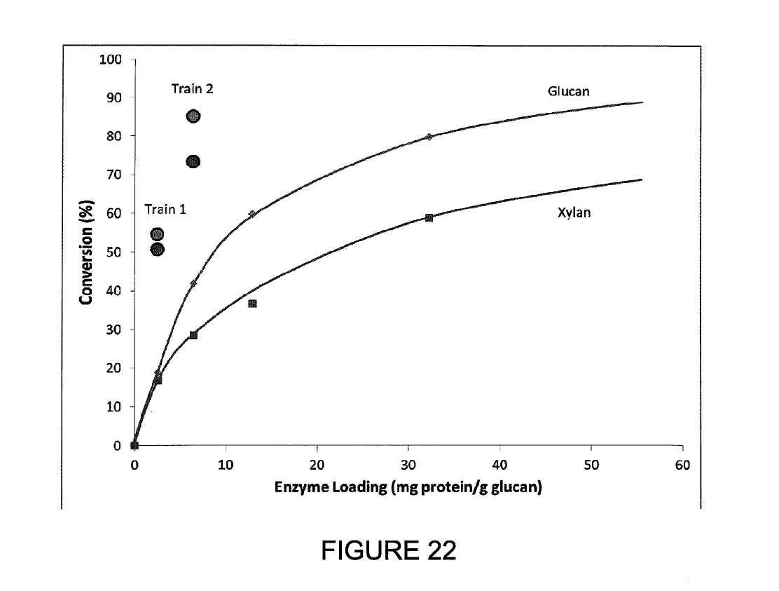

FIG. 22 illustrates example conversions achieved in continuous countercurrent saccharification to batch according to this disclosure. Table 6 also compares the conversions.

TABLE 6

| | Train 1 | Train 2 |
|---|---|---|
| Glucose conversion (%) | 53.7 | 85.3 |
| Enzyme loading (mg protein/g glucan) | | |
| Continuous countercurrent | 2.58 | 6.46 |
| Batch | 10 | 45 |
| Factor improvement | 3.9 | 7.0 |
| Xylose conversion (%) | 50.9 | 73.0 |
| Enzyme loading (mg protein/g glucan) | | |
| Continuous countercurrent | 2.58 | 6.46 |
| Batch | 22 | 70 |
| Factor improvement | 8.5 | 10.8 |

In this example, to achieve the same glucan conversion, continuous countercurrent saccharification reduces enzyme requirements by 3.9 to 7.0 times. To achieve the same xylan conversion, continuous countercurrent saccharification reduces enzyme requirements by 8.5 to 10.8 times

Various embodiments of this disclosure described above may provide certain technical advantages depending on the implementation. Some of these technical advantages include:

the ability to achieve the same conversion as conventional systems while reducing enzyme requirements by a large factor (such as by a factor of 3.9 to 10.8), which can yield a major cost reduction because enzyme costs are significant;

the ability to introduce an enzyme at an intermediate stage in the countercurrent system, which allows it to be carried in both directions (such as when unadsorbed enzyme travels with the liquid and adsorbed enzyme travels with the solids);

the ability to allow undigested solids to be washed countercurrently to recover interstitial sugars;

the ability to incorporate antimicrobial agents into the saccharification solution and then be removed and recycled using appropriate methods, depending upon the nature of the antimicrobial agent;

the ability to create synergies by using volatile antimicrobial agents that may be removed in a sugar concentrator that employs thermal methods, which allows the same process to be used to concentrate the sugars and remove the volatile antimicrobial agents;

the ability to add antimicrobial agents directly in the form of plants (such as oregano or other plants with essential oils) without the need to extract the essential oils;

the ability to use a packed column to achieve countercurrent saccharification, while maintaining a portion of the solids in the column above the waterline to help keep the biomass from floating in the column;

the ability to thermally concentrate sugar solutions;

the ability to incorporate the concepts disclosed herein with those disclosed in U.S. Patent Publication No. 2012/0118722 (which is hereby incorporated by reference in its entirety);

the ability to use boiler ash to pretreat the biomass under alkaline conditions; and

the ability to synergize the countercurrent saccharification with shock pretreatment, which is particularly effective at low enzyme loadings.

Note that while a number of technical advantages are provided above, any particular implementation of the saccharification process 100 could provide none, one, some, or all of these technical advantages.

It will be understood that well-known processes have not been described in detail and have been omitted for brevity. Although specific steps, structures, and materials may have been described above, this disclosure may not be limited to these specifics, and others may be substituted as it is well understood by those skilled in the art, and various steps may not necessarily be performed in the sequences shown.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A saccharification process comprising:
obtaining lignocellulosic biomass; and
converting the lignocellulosic biomass into sugars by:
flowing the lignocellulosic biomass countercurrently to a flow of water through a saccharification vessel comprising a column; and
converting the lignocellulosic biomass into a sugar solution using an enzyme in the column.

2. The saccharification process of claim 1, wherein a portion of the lignocellulosic biomass extends above a water level within the column to keep the lignocellulosic biomass from floating within the column

3. The saccharification process of claim 1, further comprising:
introducing the enzyme into the column at an intermediate position between a top and a bottom of the column.

4. The saccharification process of claim 1, further comprising:
countercurrently washing undigested solids received from the column to recover interstitial sugars from the undigested solids.

5. The saccharification process of claim 1, further comprising:
adding one or more antimicrobial agents to the saccharification vessel; and
removing at least a portion of the one or more antimicrobial agents from the sugar solution.

6. The saccharification process of claim 5, further comprising:
thermally concentrating the sugar solution;
wherein removing at least the portion of the one or more antimicrobial agents from the sugar solution comprises removing at least the portion of the one or more antimicrobial agents from the sugar solution while thermally concentrating the sugar solution.

7. The saccharification process of claim 5, wherein adding the one or more antimicrobial agents comprises adding plant material having one or more essential oils to the saccharification vessel, the one or more antimicrobial agents comprising the one or more essential oils.

8. The saccharification process of claim 1, further comprising:
pretreating the lignocellulosic biomass prior to converting the lignocellulosic biomass into the sugars;
wherein pretreating the lignocellulosic biomass comprises using at least one alkali obtained from boiler ash.

9. A saccharification process comprising:
obtaining lignocellulosic biomass; and
converting the lignocellulosic biomass into sugars by:
flowing the lignocellulosic biomass countercurrently to a flow of water through multiple saccharification vessels comprising a series of stirred tanks;
separating solids exiting each stirred tank from sugars exiting each stirred tank; and
converting the lignocellulosic biomass into a sugar solution using an enzyme in at least some of the stirred tanks.

10. The saccharification process of claim 9, further comprising:
introducing the enzyme into an intermediate one of the stirred tanks.

11. The saccharification process of claim 9, further comprising:
countercurrently washing undigested solids received from a last of the stirred tanks to recover interstitial sugars from the undigested solids.

12. The saccharification process of claim 9, further comprising:
adding one or more antimicrobial agents to at least one of the stirred tanks; and
removing at least a portion of the one or more antimicrobial agents from the sugar solution.

13. The saccharification process of claim 12, further comprising:
thermally concentrating the sugar solution;
wherein removing at least the portion of the one or more antimicrobial agents from the sugar solution comprises removing at least the portion of the one or more antimicrobial agents from the sugar solution while thermally concentrating the sugar solution.

14. The saccharification process of claim 12, wherein adding the one or more antimicrobial agents comprises adding plant material having one or more essential oils to at least one of the stirred tanks, the one or more antimicrobial agents comprising the one or more essential oils.

15. The saccharification process of claim 9, further comprising:
pretreating the lignocellulosic biomass prior to converting the lignocellulosic biomass into the sugars;
wherein pretreating the lignocellulosic biomass comprises using at least one alkali.

16. The saccharification process of claim 15, further comprising:
obtaining the alkali from boiler ash.

17. A saccharification process comprising:
obtaining lignocellulosic biomass; and
converting the lignocellulosic biomass into sugars by:
adding one or more antimicrobial agents to the lignocellulosic biomass;
flowing the lignocellulosic biomass countercurrently to a flow of water through at least one saccharification vessel;
converting the lignocellulosic biomass into a sugar solution using an enzyme in the at least one saccharification vessel;
concentrating the sugar solution; and
removing at least a portion of the one or more antimicrobial agents from the sugar solution while concentrating the sugar solution.

18. The saccharification process of claim 17, wherein:
the one or more antimicrobial agents comprise a volatile antimicrobial agent; and
concentrating the sugar solution and removing at least the portion of the one or more antimicrobial agents from the sugar solution comprises evaporating the sugar solution.

19. The saccharification process of claim 17, further comprising:
evaporating the sugar solution using multiple evaporator stages and multiple heat exchangers.

20. The saccharification process of claim 19, wherein:
at least one of the heat exchangers comprises multiple heat exchanger tubes; and
each heat exchanger tube comprises grooves extending along at least a portion of the heat exchanger tube.

21. The saccharification process of claim 20, wherein at least one of the heat exchanger tubes has a hydrophobic coating.

22. The saccharification process of claim 19, wherein each evaporator stage comprises:
a first jet ejector configured to create a circulating flow associated with a first side of the evaporator stage; and
a second jet ejector and a pump configured to create a circulating liquid associated with a second side of the evaporator stage.

23. The saccharification process of claim 19, wherein:
evaporating the sugar solution comprises generating steam to be purged from a vapor-compression system, the vapor-compression system comprising the evaporator stages and the heat exchangers; and
providing the purged steam to one or more multi-effect evaporators.
